(19) United States
(12) Patent Application Publication
(10) Pub. No.: US 2023/0106110 A1
(43) Pub. Date: Apr. 6, 2023

(54) DENTAL APPLIANCE AND METHOD OF USE FOR DENTAL ARCH AND/OR AIRWAY DEVELOPMENT

(71) Applicant: Martin Gorman, Encino, CA (US)

(72) Inventor: Martin Gorman, Encino, CA (US)

(21) Appl. No.: 17/959,965

(22) Filed: Oct. 4, 2022

Related U.S. Application Data
(60) Provisional application No. 63/251,810, filed on Oct. 4, 2021.
(60) Provisional application No. 63/266,899, filed on Jan. 18, 2022.

(51) Int. Cl. A61F 5/56; A61C 7/10; A61C 7/08

(52) U.S. Cl. CPC A61F 5/566; A61C 7/10; A61C 7/08

(57) ABSTRACT

A succession of dental appliances may be used to provide airway remodeling. The airway remodeling may provide for treatment of snoring or sleep disordered breathing. The dental appliances may have an outside wall, an inside wall, and a channel to receive a dental arch between the inside wall and the outside wall. The inside wall may extend so as to apply pressure to a bone structure, when worn by a user. The succession of dental appliances may have successively greater arch widths and/or lengths.

### CROSS-REFERENCE TO RELATED APPLICATIONS

This application claims the benefit of the filing dates of U.S. Provisional Patent Application No. 63/251,810, filed on Oct. 4, 2021, and U.S. Provisional Patent Application No. 63/266,899, filed on Jan. 18, 2022, the disclosures of which are incorporated by reference herein.

### FIELD OF THE INVENTION

The present invention relates generally to dental appliances, and more specifically to dental appliances and methods for jaw and pharyngeal airway remodeling and/or treatment of snoring and sleep disordered breathing.

### BACKGROUND OF THE INVENTION

Narrow pharyngeal and nasal airways and temporary obstructions of the pharyngeal airway by other portions of the anatomy, for example the tongue, may cause a host of issues. Narrow pharyngeal airways may result in reduction of inhalation of oxygen and exhalation of carbon dioxide. Narrow pharyngeal airways may also increase the likelihood of temporary pharyngeal airway obstruction, for example by the tongue, during sleep. Such airway obstruction may result in sleep disordered breathing, and possibly a host of ill effects associated with sleep disordered breathing.

Surgical procedures for overly narrow pharyngeal airways may be available. However, performance of such procedures may not result in successful outcomes, and surgical procedures generally have concomitant risks and possible complications.

Previously known dental appliances which modify jaw positions and dental arches may be considered, for example instead of surgery. Such dental appliances, however, may be bulky and/or uncomfortable for patients. Moreover, their use may also result in misalignment or mis-positioning of tooth locations within the mouth, necessitating subsequent additional efforts to correct any teeth misalignment or mis-positioning.

### BRIEF SUMMARY OF THE INVENTION

Some aspects of the invention provide a plurality of dental appliances, each dental appliance including an outside wall and an inside wall, with a channel to receive a dental arch between the outside wall and the inside wall, the inside wall extending, in a same direction as the channel, sufficiently far to be parallel to at least one bone structure supporting teeth of the dental arch, so as to apply pressure to the at least one bone structure in a direction towards a surface defined by the outside wall, each of the plurality of dental appliances having inside walls having different arch widths and/or lengths. In some aspects the dental appliances may be arranged in a succession of dental appliances, each dental appliance in the succession having a greater arch width and/or length than a prior dental appliance in the succession. In some embodiments each dental appliance in the succession has an arch width and/or length between approximately 0.17 to 0.25 millimeters greater than an arch width and/or length of an immediately preceding dental appliance in the succession. In some embodiments each dental appliance in the succession has an arch width and/or length approximately 0.17 millimeters greater than an arch width and/or length of an immediately preceding dental appliance in the succession. In some embodiments each dental appliance in the succession has an arch width and/or length approximately 0.25 millimeters greater than an arch width and/or length of an immediately preceding dental appliance in the succession. In some embodiments one of the dental appliances is a maxillary dental appliance and another of the dental appliances is a mandibular dental appliance. In some embodiments the maxillary dental appliance includes a surface extending across the dental arch, with the surface configured so as to be about a palate of an individual who is to wear the maxillary dental appliance. In some embodiments the surface connects extends from upper edges of the inside wall of the maxillary dental appliance. In some embodiments the surface connects the upper edges of the inside wall of the maxillary dental appliance. In some embodiments the surface covers an area within an arch formed by the upper edges of the inside wall of the maxillary dental appliance. In some embodiments the surface of each maxillary dental appliance in the succession of dental appliances is configured, at least in part, to be 0.25 to 1.0 mm below the palate of the individual who is to wear the maxillary dental appliance, at the time the individual is to begin wearing the maxillary dental appliance. In some embodiments the surface of each maxillary dental appliance in the succession of dental appliances is configured, at least in part, to be 0.25 mm below the palate of the individual who is to wear the maxillary dental appliance, at the time the individual is to begin wearing the maxillary dental appliance. In some embodiments the surface of each maxillary dental appliance in the succession of dental appliances is configured, at least in part, to be 1.0 mm below the palate of the individual who is to wear the maxillary dental appliance, at the time the individual is to begin wearing the maxillary dental appliance. In some embodiments an apex of the surface of each maxillary dental appliance in the succession of dental appliances is configured to be 0.25 mm below an apex of an immediately prior maxillary dental appliance in the sequence of dental appliances. In some embodiments the apex may be considered to be lowered for each successive maxillary dental appliance, to allow for greater ease of lowering the individual's palate. In some aspects the dental appliances are formed of a polymer. In some embodiments the polymer is an acrylic polymer. In some embodiments the outside wall, inside wall, and channel are integrally formed of an acrylic polymer. In some embodiments the channel includes indentations for at least some teeth of the dental arch. In some embodiments the channel includes indentations for all of the teeth of the dental arch. In some embodiments positions of the indentations for at least some of the teeth reflect a common spacing between teeth across different ones of the dental appliances.

In some embodiments the positions of the indentations for at least some of the teeth provide for orthodontic positioning of the at least some of the teeth by at least some of the different ones of the dental appliances. In some embodiments the orthodontic positioning of the teeth is provided by each dental appliance in the sequence of dental appliances.

Some aspects of the invention provide a method of increasing a width and/or length of a dental arch, comprising: providing a succession of dental appliances to a patient for wear by the patient, each dental appliance comprising a polymeric form including an outside wall and an inside wall, with a channel to receive a dental arch between the outside wall and the inside wall the inside wall extending, in a same direction as the channel, sufficiently far to be parallel to at least one bone structure supporting teeth of the dental arch, so as to apply pressure to the at least one bone structure in a direction towards a surface defined by the outside wall, each of the plurality of dental appliances having inside walls having different arch widths and/or lengths, each subsequent dental appliance in the succession having a greater arch width and/or length than a preceding dental appliance in the succession. In some embodiments the succession of dental appliances include indentations to receive teeth. In some embodiments the succession of dental appliances orthodontically position the teeth through positioning of the indentations.

These and other aspects of the invention are more fully comprehended upon review of this disclosure.

### BRIEF DESCRIPTION OF THE FIGURES

FIG. 1 shows an example cast of a maxillary dental arch and palate.

FIG. 2 shows an example cast of a mandibular dental arch.

FIG. 3 shows a lower rear perspective view of an example maxillary dental appliance.

FIG. 4 shows an upper rear perspective view of an example mandibular dental appliance.

FIG. 5A shows an upper rear perspective view of a maxillary dental appliance in accordance with aspects of the invention.

FIG. 5B shows an upper rear perspective view of a further maxillary dental appliance in accordance with aspects of the invention.

FIG. 6A shows a lower rear perspective view of a mandibular dental appliance in accordance with aspects of the invention.

FIG. 6B shows a lower rear perspective view of a further mandibular dental appliance in accordance with aspects of the invention.

FIG. 7 is a flow diagram of a process that may be used in making a sequence of dental appliances, in accordance with aspects of the invention.

FIG. 8 shows an upper rear perspective view of a further maxillary dental appliance, in accordance with aspects of the invention.

FIG. 9 shows a lower rear perspective view of the example maxillary dental appliance of FIG. 8, with a representation of a hook attached about a position a first molar.

FIG. 10 shows a lower rear perspective view of a further mandibular dental appliance, in accordance with aspects of the invention.

FIG. 11 shows an upper rear perspective view of the example mandibular dental appliance of FIG. 10, with a representation of a hook attached about a position a first molar.

FIG. 12 diagrammatically shows a facemask on a head of a patient, in accordance with aspects of the invention.

### DETAILED DESCRIPTION

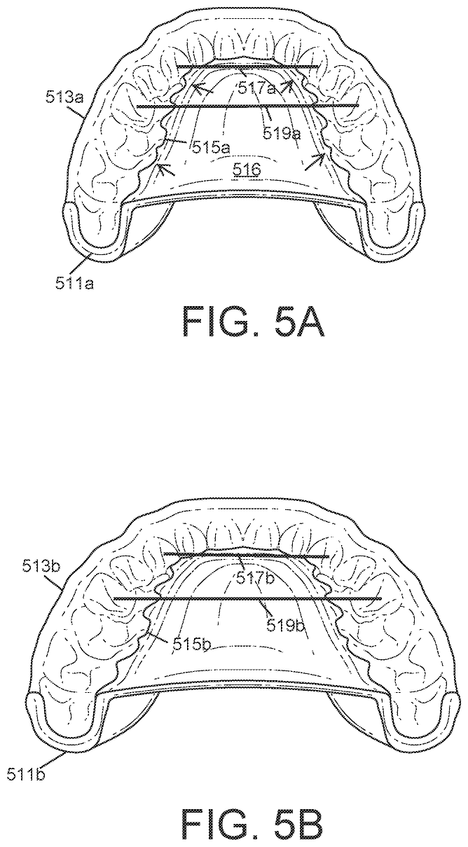

FIG. 5A shows an upper rear perspective view of a maxillary dental appliance in accordance with aspects of the invention. A generally U-shaped channel 511a is defined on its sides by an outside wall 513a and an inside wall 515a. The U-shaped channel generally follows a path of a dental arch, for example of a patient, and the channel may include indentations to receive maxillary teeth of the patient. In FIG. 5A, a surface 516 connects the inside wall, across the dental arch. The surface may for example extend from upper edges of the inside wall. The surface may, for example, conform to a palate of the patient, and be positioned so as to be adjacent the palate when the dental appliance is worn by the patient. Accordingly, as a palate of a patient will generally curve upward away from the jaw, towards an apex, the surface in many embodiments also curves upward towards an apex with distance from the inner wall. In some embodiment the surface may be considered to cover (from below) the palate of the patient in the area within an arch formed by the inside wall. In some embodiments the surface may generally conform in shape to the palate of the patient, but be offset by a distance, for example 0.25 mm, 1.0 mm, or between 0.25 and 1.0 mm, from an apex of the palate. In some embodiments the offset from the palate may be at a maximum along an apex of the palate, which may be for example along a mid-line of the palate generally equidistant from opposing molars on opposing sides of the dental arch.

The inside wall extends upward sufficiently far so as to be parallel to the upper jawbone. In some embodiments the inside wall is in contact with the palatal dome tissue. In some embodiments at least part of the inside wall is in contact with a vertical portion of the palatal dome tissue. Preferably, the inside wall is configured to apply pressure to the upper jawbone, in an outward direction such that a width and/or length of the dental arch may be increased. Concomitantly, the outside wall may be slightly forward than would be expected for a close fit for the maxillary teeth, allowing for movement of the teeth as the dental arch increases.

The device of FIG. 5A may, for example, be one of a sequence of dental appliances. In some embodiments, each successive dental appliance in the sequence may include an inside wall that is farther forward for example by the directions of arrows in FIG. 5A, than an immediately prior dental appliance in the sequence. In some embodiments the outside wall may be farther forward by an equivalent amount, with the channel also therefore being farther forward. In some embodiments the inside wall may be farther forward in a range of 0.1 to 0.5 mm. In some embodiments the inside wall may be farther forward in a range of 0.15 to 0.3 mm. In some embodiments the inside wall may be farther forward in a range of 0.18 to 0.25 mm. In some embodiments the inside wall may be farther forward in a range of 0.16 to 0.20 mm. In some embodiments the inside wall may be farther forward in a range of 0.23 to 0.27 mm. In some embodiments the surface conforming to, or slightly offset from, the palate, or in many embodiments an apex of the surface, is also slightly lower for each successive dental appliance in the sequence. In some embodiments the surface may be 0.25 to 1.0 mm lower for each successive dental appliance, for example to accommodate lowering of the palate with use of each successive dental appliance. In some embodiments the surface may be 0.25 mm lower for each successive dental appliance, for example to accommodate lowering of the palate with use of each successive dental appliance. Successive use of the dental appliances, therefore, may result in increase in width and/or length of the dental arch. In some embodiments the dental appliances include indentations for teeth. In some embodiments positions of the indentations for the teeth are determined for at least some of the dental appliances in the sequence of dental appliances so as to orthodontically position the teeth.

FIG. 5B shows an upper rear perspective view of a further maxillary dental appliance in accordance with aspects of the invention. The further maxillary dental appliance of FIG. 5B may be a dental appliance in the same sequence of dental appliances as that of FIG. 5A, and in some embodiments may be an ultimate dental appliance in that sequence. The further maxillary dental appliance of FIG. 5B is substantially the same as the dental appliance of FIG. 5A, with a channel 511b, defined by an outside wall 513b and an inside wall 515b, to accept teeth of a maxillary dental arch. As with the dental appliance of FIG. 5A, the inside wall of the dental appliance of FIG. 5B is configured to apply pressure to the upper jawbone in an outward direction.

For the dental appliance of FIG. 5B, however, the dental arch is differently positioned. The dental appliance of FIG. 5B is for a dental arch that has a greater width and/or length, a greater width and/or length due to increases in width and/or length caused by use of one or more prior dental appliances in the sequence. In addition, in some embodiments the surface 516 connecting the inside wall across the dental arch of FIG. 5B also has a lower apex than that of FIG. 5A, for example so as to allow for lowering of a palate of a user of the dental arch.

In FIGS. 5A and 5B, the increase in dental arch width is illustrated, among other ways, in differences in intercanine width and second interpremolar width. In FIG. 5A, intercanine width 517a is a first distance, while in FIG. 5B, intercanine width 517b is a second distance, the second distance being greater than the first distance. Similarly, for the dental appliance of FIG. 5A, a second interpremolar width 519a is less than a second interpremolar width 519b of the dental appliance of FIG. 5B.

In some embodiments the position of indentations in the channel for the teeth may also be comparatively adjusted between dental appliances in the sequence. Absent comparative adjustment of the indentations for the teeth, as the dental arch increases in width and/or length, distance between the teeth would also increase. With adjustment of the position of indentations for the teeth, however, the teeth may also be orthodontically positioned as desired.

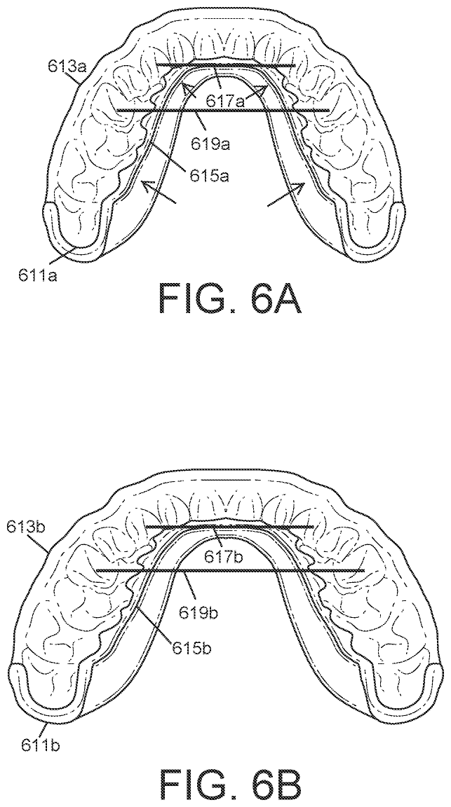

FIG. 6A shows a lower rear perspective view of a mandibular dental appliance in accordance with aspects of the invention. As with the maxillary dental appliance, A generally U-shaped channel 611a is defined on its sides by an outside wall 613a and an inside wall 615a. The U-shaped channel generally follows a path of a dental arch, for example of a patient, and the channel may include indentations to receive mandibular teeth of the patient.

The inside wall extends downward sufficiently far so as to be parallel to the portion of the body of the mandible holding the mandibular teeth. Preferably, the inside wall is sufficiently forward so as to apply pressure to the bone, in an outward direction such that a width and/or length of the dental arch is increased. In some embodiments the inside wall extends below a gumline for the mandibular teeth. In some embodiments the inside wall extends 4 to 5 mm below the gumline for the mandibular teeth. Concomitantly, the outside wall may be slightly forward than would be expected for a close fit for the mandibular teeth, allowing for movement of the teeth as the dental arch increases.

As with the dental appliance of FIG. 5A, the device of FIG. 6A may, for example be one of a sequence of dental appliances. In some embodiments, each successive dental appliance in the sequence may include an inside wall that is farther forward for example by the directions of arrows in FIG. 6A, than an immediately prior dental appliance in the sequence. In some embodiments the outside wall may be farther forward by an equivalent amount, with the channel also therefore being farther forward. In some embodiments the inside wall may be farther forward in a range of 0.1 to 0.5 mm. In some embodiments the inside wall may be farther forward in a range of 0.15 to 0.3 mm. In some embodiments the inside wall may be farther forward in a range of 0.18 to 0.25 mm. In some embodiments the inside wall may be farther forward in a range of 0.16 to 0.20 mm. In some embodiments the inside wall may be farther forward in a range of 0.23 to 0.27 mm. Successive use of the dental appliances, therefore, may result in increase in width and/or length of the dental arch. In addition, in some embodiments the dental appliances include indentations for teeth, and positions of the indentations for the teeth may orthodontically position the teeth as the width and/or length of the dental arch is modified.

FIG. 6B shows a lower rear perspective view of a further mandibular dental appliance in accordance with aspects of the invention. The further mandibular dental appliance of FIG. 6B may be a dental appliance in the same sequence of dental appliances as that of FIG. 6A, and in some embodiments may be an ultimate dental appliance in that sequence. The further mandibular dental appliance of FIG. 5B is substantially the same as the dental appliance of FIG. 5A, with a channel 611b, defined by an outside wall 613b and an inside wall 615b, to accept teeth of a mandibular dental arch. As with the dental appliance of FIG. 6A, the inside wall of the dental appliance of FIG. 6B extends sufficiently far so as to apply pressure to the mandibular bone in an outward direction.

For the dental appliance of FIG. 6B, however, the dental arch is differently positioned. The dental appliance of FIG. 6B is for a dental arch that has a greater width and/or length, a greater width and/or length due to increases in width and/or length caused by use of one or more prior dental appliances in the sequence.

In FIGS. 6A and 6B, the increase in dental arch width is illustrated, among other ways, in differences in intercanine width and second interpremolar width. In FIG. 6A, intercanine width 617a is a first distance, while in FIG. 6B, intercanine width 617b is a second distance, the second distance being greater than the first distance. Similarly, for the dental appliance of FIG. 6A, a second interpremolar width 619a is less than a second interpremolar width 619b of the dental appliance of FIG. 6B.

In some embodiments the position of indentations in the channel for the teeth may also be comparatively adjusted between dental appliances in the sequence. Absent comparative adjustment of the indentations for the teeth, as the dental arch increases in width and/or length, distance between the teeth would also increase. With adjustment of the position of indentations for the teeth, however, the teeth may also be orthodontically positioned as desired.

In some embodiments the sequence of dental appliances discussed with respect to FIGS. 5A and 5B and FIGS. 6A and 6B, respectively, may be used together, with the width and/or length of the upper and lower dental arches being moved in unison.

In some embodiments the dental appliances of FIGS. 5A, 5B, 6A, 6B, and other dental appliances in a sequence of such dental appliances is formed of a polymer. In some embodiments the dental appliances are formed of an acrylic. In some embodiments the dental appliances are formed of an acrylic resin.

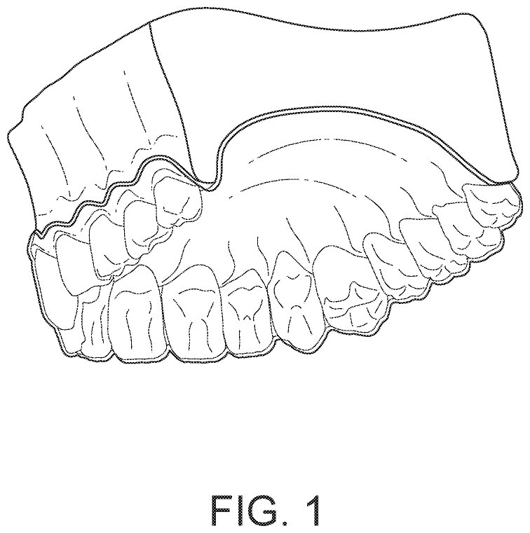

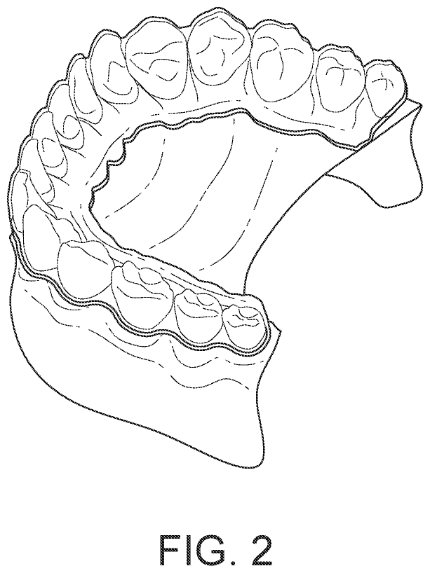

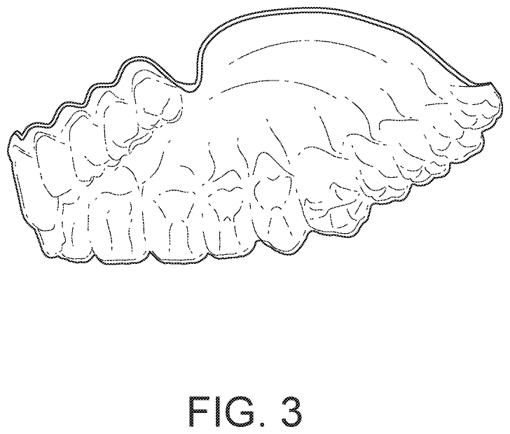

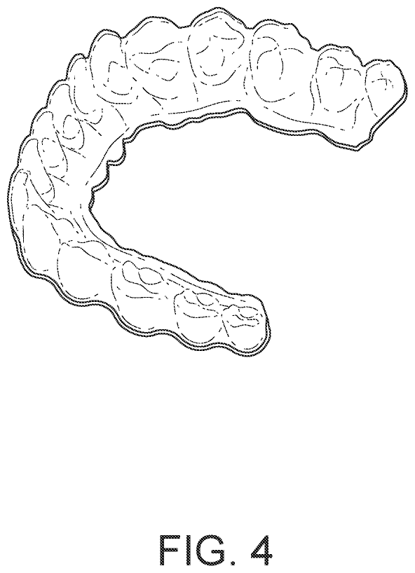

In some embodiments the dental appliances are formed using casts of a patient's maxillary dental arch and mandibular dental arch. FIG. 1, for example, shows a cast of a maxillary dental arch and palate, and FIG. 2, for example, shows a cast of a mandibular dental arch. The dental appliances may be formed using the casts of the dental arches, or information of the casts. In some embodiments a dental appliance is made using the casts, but extending the inside and outside walls of the grooves. In some embodiments the inside and outside wall may be extended between 0.15 and 0.30 mm. In some embodiments the inside and outside wall may be extended between 0.18 and 0.25 mm. In some embodiments the inside and outside wall may be extended by 0.18 mm. In some embodiments the inside and outside wall may be extended by 0.25 mm. FIG. 3 shows a lower rear perspective view of an example maxillary dental appliance, and FIG. 4 shows an upper rear perspective view of an example mandibular dental appliance. After the dental appliances are used for a period of time, one week in some embodiments, a period of time expected to result in 120 hours of patient use of the dental appliances in some embodiments, further casts may be made for use in making subsequent dental appliances in a sequence of dental appliances.

In some embodiments information provided by the casts, or for example by imaging of teeth, and possibly bone positions in some embodiments, may be used in making an entire sequence of dental appliances.

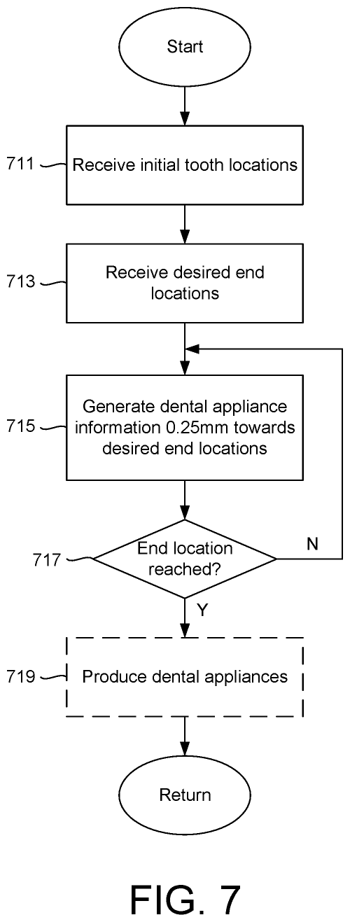

FIG. 7 is a flow diagram of a process that may be used in making a sequence of dental appliances. The dental appliances may be the sequence of dental appliances discussed with respect to FIGS. 5A-6B. In some embodiments the process is performed, at least in part, by a computer. In some embodiments dental appliance production information is generated using a computer. In some embodiments the computer includes at least one processor, associated memory, and at least one user input device. In some embodiments the computer is a server. In some embodiments the computer is coupled to a network, for example a local area network and/or the Internet, and different computers and/or servers may perform different parts of the process.

In block 711 the process receives information of initial tooth and/or dental arch locations for a patient. The information of initial tooth and/or dental arch locations may be derived from a cast of the patient's dental arches, for example, in some embodiments the information of initial tooth and/or dental arch locations may be derived from imaging of the patient's dental arches. In some embodiments the information includes information of palate location/position, or of an apex of the palate, for some or all locations within the dental arch.

In block 713 the process receives information of desired tooth and/or dental arch locations. The desired locations may be desired tooth and/or dental arch locations after use of a sequence of dental appliances to increase width and/or length of the patient's dental arches. In some embodiments the process may also receive a desired location or locations for an apex of the palate of the patient.

In block 715 the process generates information of a dental appliance to increase width and/or length of the patient's dental arches by a predetermined amount. In some embodiments the predetermined amount is between 0.15 and 0.30 mm. In some embodiments the predetermined amount is between 0.18 and 0.25 mm. In some embodiments the predetermined amount is 0.18 mm. In some embodiments the predetermined amount is 0.25 mm. In some embodiments a direction of increase is determined based differences between a current position and a desired position for same portions of the dental arches. In some embodiments positions of the portions are based on positions of the same teeth. In some embodiments the generated information includes information of position of a surface connecting the dental arch of the upper or maxillary dental appliance, or an apex of the surface.

In block 717 the process determines if the information of the dental appliance of block 715 indicates that the desired end locations of the tooth and/or dental arch locations have been reached, or, in some embodiments, are within a predetermined range of being reached. If not, the process returns to operations of block 715, and generates a further dental appliance in the sequence, with the location information of teeth and/or dental arches presumed to be those locations as adjusted by use of a dental appliance produced in accordance with the information of the prior dental appliance generated in the immediately prior operations of block 715. If the desired end locations have been reached, the process continues to block 719.

In block 719, dental appliances are produced based the information generated in successive operations of block 715. In some embodiments operations of block 719 are performed outside of the process of FIG. 7, for example as part of another process. In some embodiments the operations of block 719 are performed by a molding machine, or some other machine.

The process thereafter returns.

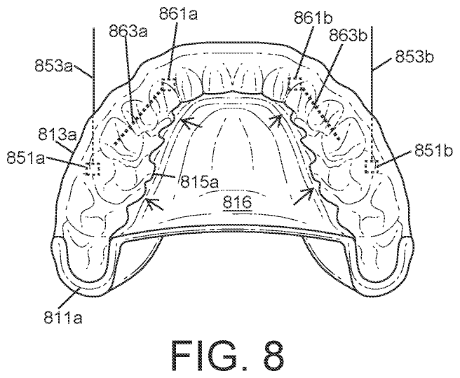

FIG. 8 shows an upper rear perspective view of a further maxillary dental appliance in accordance with aspects of the invention. The maxillary dental appliance of FIG. 8 generally mirrors that of FIG. 5A, with the exception that the appliance of FIG. 8 includes hooks 851a,b for holding elastic bands 853a,b attached to a facemask, for example to apply forward pressure to the dental appliance. The appliance also includes hooks 861a,b for holding elastic bands 863a,b attached to a mandibular dental appliance for providing class II elastics.

As in FIG. 5A, the dental appliance of FIG. 8 has a generally U-shaped channel 811a defined on its sides by an outside wall 813a and an inside wall 815a. The U-shaped channel generally follows a path of a dental arch, for example of a patient, and the channel may include indentations to receive maxillary teeth of the patient. A surface 816 connects the inside wall, across the dental arch. The surface may, for example, conform to a palate of the patient, and be positioned so as to be adjacent or offset from the palate, or an apex of the palate, when the dental appliance is worn by the patient.

The inside wall extends upward sufficiently far so as to be parallel to the upper jawbone, and in some embodiments in contact with the palatal dome tissue of the upper jawbone. In some embodiments the inside wall is in contact with a vertical portion of the palatal dome tissue. In some embodiments the inside wall extends upwards so as to be above a gumline, in some embodiments above the gumline by 4 to 5 mm. Preferably, the inside wall is configured to apply pressure to the upper jawbone, in an outward direction such that a width and/or length of the dental arch may be increased. Concomitantly, the outside wall may be slightly forward than would be expected for a close fit for the maxillary teeth, allowing for movement of the teeth as the dental arch increases.

The device of FIG. 8 may be one of a sequence of dental appliances, for example as discussed with respect to FIG. 5A. As discussed with respect to FIG. 5A, in some embodiments, each successive dental appliance in the sequence may include an inside wall that is farther forward for example by the directions of arrows in FIG. 8, than an immediately prior dental appliance in the sequence. In some embodiments the outside wall may be farther forward by an equivalent amount, with the channel also therefore being farther forward. In addition, the surface connecting opposing sides of the dental arch, or the apex of the surface, may be lowered in each successive dental appliance, to allow for lowering of the patient's palate. Successive use of the dental appliances, therefore, may result in increase in width and/or length of the dental arch.

The hooks 851a,b are mounted to the exterior of opposing sides of the outer wall. As illustrated in FIG. 8, the hooks are mounted about positions of the first molar, although the hooks may be positioned about other tooth locations in various embodiments. Elastic bands 853a,b are coupled to the hooks 851a,b, respectively. The elastic bands extend outwardly past a front of the dental appliance, for coupling to a cross-bar or other portion of a facemask to be worn by the patient. The elastic bands are preferably under tension, so as to transmit a forward force to the dental appliance as a whole by way of the hooks.

The hooks 861a,b are also mounted to the exterior of opposing sides of the outer wall. As illustrated in FIG. 8, the hooks are mounted about positions of the canines, although the hooks may be positioned about other tooth locations in various embodiments. Elastic bands 863a,b are coupled to the hooks 861a,b, respectively. The elastic bands extend rearwardly and slightly downward, for coupling to hooks on an outer wall of a mandibular dental appliance. The hooks on the mandibular dental appliance may be about positions of the lower molars, although the hooks may be positioned about other tooth locations in various embodiments. The use of the hooks 861a,b and elastic bands 863a,b may be used in providing class II elastics, and may serve to temporarily move the mandible of the patient forward, to possibly reduce snoring and possibly provide for increased opening of the patient's airway, at least temporarily.

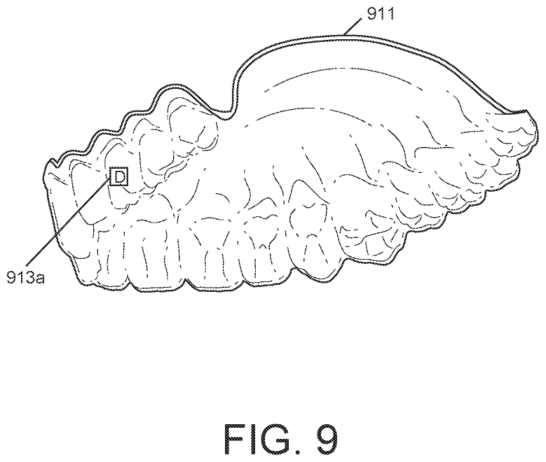

FIG. 9 shows a lower rear perspective view of the example maxillary dental appliance 911 of FIG. 8, with a representation of a hook 913a attached about a position a first molar. A corresponding hook would be attached on an opposing side of the dental appliance, about a position of the opposing first molar. Further hooks may be attached about positions of the canines, for example for use in providing class II elastics.

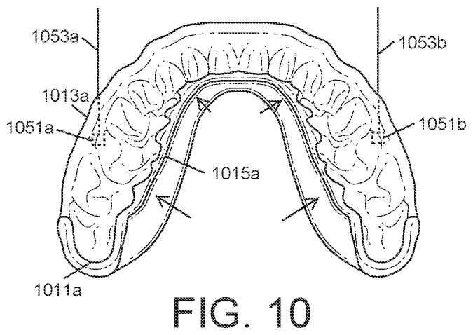

FIG. 10 shows a lower rear perspective view of a further mandibular dental appliance in accordance with aspects of the invention. The mandibular dental appliance of FIG. 10 generally mirrors that of FIG. 6A, with the exception that the appliance of FIG. 10, like that of FIG. 8, includes hooks 1051a,b for holding elastic bands 1053a,b attached to hooks on the maxillary dental appliance for use in providing class II elastics, for example to apply forward pressure to the mandibular dental appliance.

As with the maxillary dental appliance of FIG. 6A, the dental appliance of FIG. 10 generally U-shaped channel 1011a is defined on its sides by an outside wall 1013a and an inside wall 1015a. The U-shaped channel generally follows a path of a dental arch, for example of a patient, and the channel may include indentations to receive mandibular teeth of the patient.

The inside wall extends downward sufficiently far so as to be parallel to the portion of the body of the mandible holding the mandibular teeth. Preferably, the inside wall is sufficiently forward so as to apply pressure to the bone, in an outward direction such that a width and/or length of the dental arch. In some embodiments the inside wall extends below a gumline for the mandibular teeth. In some embodiments the inside wall extends 4 to 5 mm below the gumline for the mandibular teeth. Concomitantly, the outside wall may be slightly forward than would be expected for a close fit for the mandibular teeth, allowing for movement of the teeth as the dental arch increases.

As with the dental appliances of FIGS. 5A and 6A, the dental appliance of FIG. 10 may, for example be one of a sequence of dental appliances, for example as discussed with respect to FIGS. 5A and 6A. In some embodiments, each successive dental appliance in the sequence may include an inside wall that is farther forward for example by the directions of arrows in FIG. 10, than an immediately prior dental appliance in the sequence. In some embodiments the outside wall may be farther forward by an equivalent amount, with the channel also therefore being farther forward. Successive use of the dental appliances, therefore, may result in increase in width and/or length of the dental arch.

The hooks 1051a,b are mounted to the exterior of opposing sides of the outer wall. As illustrated in FIG. 10, the hooks are mounted about positions of the first molar, although the hooks may be positioned about other tooth locations in various embodiments. Elastic bands 1053a,b are coupled to the hooks 1051a,b, respectively. The elastic bands extend outwardly towards a front of the mandibular dental appliance, and slightly upward, for coupling to hooks on a maxillary dental appliance, for example about positions of the canines. The hooks and elastic bands may be used in providing a class II elastic. The elastic bands are preferably under tension, so as to transmit a forward force to the dental appliance as a whole by way of the hooks.

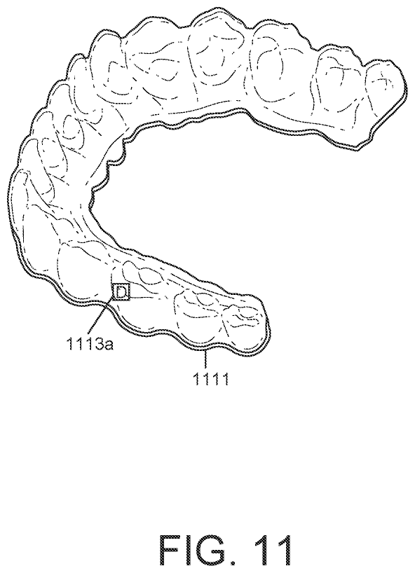

FIG. 11 shows an upper rear perspective view of the example mandibular dental appliance 1011 of FIG. 10, with a representation of a hook 1113a attached about a position a first molar. A corresponding hook would be attached on an opposing side of the dental appliance, about a position of the opposing first molar.

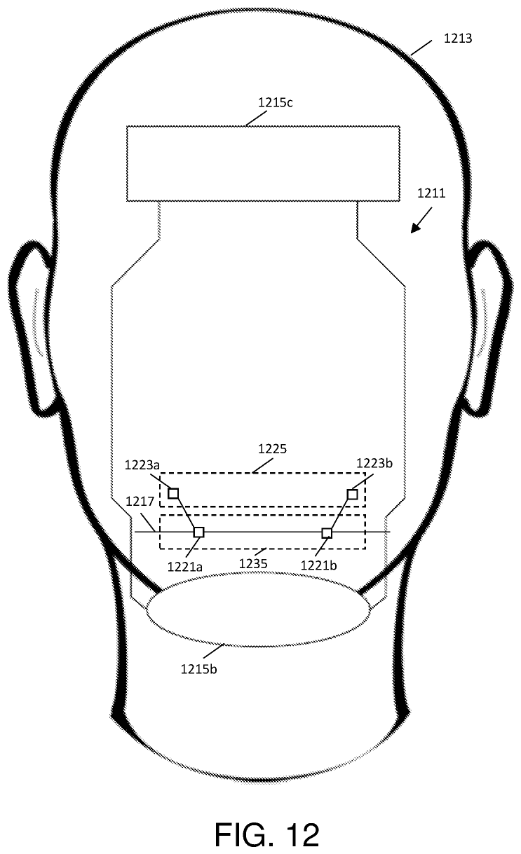

For completeness, FIG. 12 diagrammatically shows a facemask 1211 on a head 1213 of a patient. The facemask includes an upper pad 1215a for placement against a forehead of the patient, and a lower pad 1215b for placement against a chin of the patient. Generally vertical bars couple the upper pad the lower pad, about sides of the patient's face. A horizontal cross-bar 1217 couples the vertical bars, with the cross-bar positioned somewhat below a level of the patient's mouth. In various embodiments the cross-bar is positioned approximately 15-20 degrees below a level, or horizon, defined by the patient's mouth.

A pair of couplings 1221a,b are coupled to the cross-bar. Elastic bands extend between each of the couplings and corresponding hooks 1223a,b on a maxillary dental appliance 1225. The maxillary dental appliance 1225 and a mandibular dental appliance 1235 may be as discussed with respect to FIGS. 8-11. The elastic bands are preferably under tension, so as to both press the facemask against the forehead and chin of the patient, and to pull forward the maxillary and dental appliance. In some embodiments the elastic bands may be coupled directly to the cross-bar.

Although the invention has been described with respect to certain embodiments, it should be recognized that the invention comprises the novel and nonobvious claims supported by this disclosure.

What is claimed is:

1. Dental appliances for jaw and pharyngeal airway remodeling and/or treatment of snoring and sleep disordered breathing, comprising:
a plurality of dental appliances, each dental appliance including an outside wall and an inside wall, with a channel to receive a dental arch between the outside wall and the inside wall, the inside wall extending, in a same direction as the channel, sufficiently far to be parallel to at least one bone structure supporting teeth of the dental arch, so as to apply pressure to the at least one bone structure in a direction towards a surface defined by the outside wall;
with each of the plurality of dental appliances having inside walls having different arch widths and/or lengths.

2. The dental appliances of claim 1, wherein the dental appliances provide a succession of dental appliances, each dental appliance in the succession having a greater arch width and/or length than a prior dental appliance in the succession.

3. The dental appliances of claim 2, wherein each dental appliance in the succession has an arch width and/or length between 0.15 and 0.30 millimeters greater than an arch width and/or length of an immediately preceding dental appliance in the succession.

4. The dental appliances of claim 2, wherein the dental appliances comprise maxillary dental appliances.

5. The dental appliances of claim 4, wherein the maxillary dental appliances include a surface extending across the dental arch, with the surface configured so as to be about a palate of an individual who is to wear the maxillary dental appliances.

6. The dental appliances of claim 5, wherein the surface connects extends from upper edges of the inside wall of the maxillary dental appliance.

7. The dental appliances of claim 6, wherein the surface connects the upper edges of the inside wall of the maxillary dental appliance.

8. The dental appliances of claim 6, wherein the surface covers an area within an arch formed by the upper edges of the inside wall of the maxillary dental appliance.

9. The dental appliances of claim 6, wherein the surface of each maxillary dental appliance in the succession of dental appliances is configured, at least in part, to be between 0.25 and 1.0 mm below the palate of the individual who is to wear the maxillary dental appliance, at the time the individual is to begin wearing the maxillary dental appliance.

10. The dental appliances of claim 6, wherein an apex of the surface of each maxillary dental appliance in the succession of dental appliances is configured to be between 0.25 mm and 1.0 mm below an apex of an immediately prior maxillary dental appliance in the sequence of dental appliances.

11. The dental appliances of claim 2, wherein the dental appliances comprise mandibular dental appliances.

12. The dental appliances of claim 2, wherein the dental appliances are formed of a polymer.

13. The dental appliances of claim 12, wherein the polymer is an acrylic polymer.

14. The dental appliances of claim 13, wherein the outside wall, inside wall, and channel are integrally formed of an acrylic polymer.

15. The dental appliances of claim 2, wherein the channel includes indentations for at least some teeth of the dental arch.

16. The dental appliances of claim 15, wherein positions of the indentations for at least some of the teeth reflect a common spacing between teeth across different ones of the dental appliances.

17. The dental appliances of claim 16, wherein the positions of the indentations for at least some of the teeth provide for orthodontic positioning of the at least some of the teeth by at least some of the different ones of the dental appliances.

18. A method of increasing a width and/or length of a dental arch, comprising:
providing a succession of dental appliances to a patient for wear by the patient, each dental appliance comprising a polymeric form including an outside wall and an inside wall, with a channel to receive a dental arch between the outside wall and the inside wall the inside wall extending, in a same direction as the channel, sufficiently far to be parallel to at least one bone structure supporting teeth of the dental arch, so as to apply pressure to the at least one bone structure in a direction towards a surface defined by the outside wall, each of the plurality of dental appliances having inside walls having different arch widths and/or lengths, each subsequent dental appliance in the succession having a greater arch width and/or length than a preceding dental appliance in the succession.

19. The method of claim 18, wherein the dental appliances include indentations to receive teeth.

20. The method of claim 19, wherein the succession of dental appliances orthodontically position the teeth through positioning of the indentations.
